(19) United States
(12) Patent Application Publication
(10) Pub. No.: US 2015/0141462 A1
(43) Pub. Date: May 21, 2015

(54) PHARMACEUTICAL COMPOSITIONS AND METHODS FOR TREATING DRUG ADDICTION AND PREVENTING A DRUG RELAPSE

(71) Applicant: Arizona Board of Regents, for and on behalf of, Arizona State University, Scottsdale, AZ (US)

(72) Inventor: M. Foster Olive, Scottsdale, AZ (US)

(21) Appl. No.: 14/407,650

(22) PCT Filed: Jun. 11, 2013
(86) PCT No.: PCT/US13/45268

§ 371 Date: Dec. 12, 2014

Related U.S. Application Data
(60) Provisional application No. 61/659,379, filed on Jun. 13, 2012.

(51) Int. Cl. A61K 31/444; A61K 45/06; A61K 31/415

(52) U.S. Cl. CPC A61K 31/444; A61K 31/415; A61K 45/06
USPC 514/332; 514/407

(57) ABSTRACT

The present invention relates to pharmaceutical compositions comprising (a) a pharmaceutically effective amount of an mGluR5 positive allosteric modulator, derivative, prodrug or a pharmaceutically acceptable salt thereof; (b) a pharmaceutically effective amount of an agent selected from an NMDA partial agonist or a GlyT1 inhibitor, derivative, prodrug or a pharmaceutically acceptable salt thereof; and (c) a pharmaceutically acceptable carrier. The present invention also relates to methods for treating drug addiction and preventing a drug relapse in a patient. The methods comprise (a) administering to the patient a pharmaceutically effective amount of an mGluR5 positive allosteric modulator, derivative, prodrug or a pharmaceutically acceptable salt thereof, or a pharmaceutical composition thereof; (b) administering to the patient a pharmaceutically effective amount of an agent selected from an NMDA partial agonist or a GlyT1 inhibitor, derivative, prodrug or a pharmaceutically acceptable salt thereof, or a pharmaceutical composition thereof.

### CROSS REFERENCE TO RELATED APPLICATION

This application claims the benefit U.S. Provisional Application No. 61/659,379, filed Jun. 13, 2012, which is incorporated by reference herein in its entirety.

### FEDERALLY SPONSORED RESEARCH OR DEVELOPMENT

This invention was made with government support from grant DA024355 awarded by the National Institutes of Health. The government has certain rights in the invention.

### FIELD OF THE INVENTION

The present invention relates to pharmaceutical compositions comprising (a) a pharmaceutically effective amount of an mGluR5 positive allosteric modulator, derivative, prodrug or a pharmaceutically acceptable salt thereof; (b) a pharmaceutically effective amount of an agent selected from an NMDA partial agonist or a GlyT1 inhibitor, derivative, prodrug or a pharmaceutically acceptable salt thereof; and (c) a pharmaceutically acceptable carrier. The present invention also relates to methods for treating drug addiction and preventing a drug relapse in a patient. The methods comprise (a) administering to the patient a pharmaceutically effective amount of an mGluR5 positive allosteric modulator, derivative, prodrug or a pharmaceutically acceptable salt thereof, or a pharmaceutical composition thereof; (b) administering to the patient a pharmaceutically effective amount of an agent selected from an NMDA partial agonist or a GlyT1 inhibitor, derivative, prodrug or a pharmaceutically acceptable salt thereof, or a pharmaceutical composition thereof.

### BACKGROUND OF THE INVENTION

Drug addiction is a multifaceted disorder that places an enormous socioeconomic, legal, and medical burden on society, in addition to the destructive influences it has on the addict and his or her family and peers (Olive, M. F. 2010. Cognitive effects of Group I metabotropic glutamate receptor ligands in the context of drug addiction. European Journal of Pharmacology 639, 47-58). Current evidence suggests that drug addiction is a result of complex interactions between environmental, developmental, and genetic factors (Koob, G. F., Le Moal, M., 2007. Drug addiction: pathways to the disease and pathophysiological perspectives. Eur. Neuropsychopharmacol. 17, 377-393; Le Moal, M., 2009. Drug abuse: vulnerability and transition to addiction. Pharmacopsychiatry 42 (Suppl 1), S42-55; Spanagel, R., 2009. Alcoholism: a systems approach from molecular physiology to addictive behavior. Physiol. Rev. 89, 649-705). At the behavioral level, drug addiction is typically characterized by a transition from casual, intermittent drug use to compulsive, uncontrolled drug intake coupled with repeated failed attempts at cessation of or curtailing drug use. At the cellular and molecular levels, repeated intake of drugs of abuse produce lasting neuroadaptations in gene expression, cytoarchitecture, and synaptic plasticity in various circuitries of the brain, including the limbic, prefrontal executive control, and reward systems (Christie, M. J., 2008. Cellular neuroadaptations to chronic opioids: tolerance, withdrawal and addiction. Br. J. Pharmacol. 154, 384-396; Crews, F. T., Boettiger, C. A., 2009. Impulsivity, frontal lobes and risk for addiction. Pharmacol. Biochem. Behay. 93, 237-247; Feltenstein, M. W., See, R. E., 2008. The neurocircuitry of addiction: an overview. Br. J. Pharmacol. 154, 261-274; Kalivas, P. W., 2008. Addiction as a pathology in prefrontal cortical regulation of corticostriatal habit circuitry. Neurotox. Res. 14, 185-189; Koob, G. F., Volkow, N. D., 2010. Neurocircuitry of addiction. Neuropsychopharmacology 35, 217-238; Robbins, T. W., Arnsten, A. F., 2009. The neuropsychopharmacology of fronto-executive function: monoaminergic modulation. Annu Rev. Neurosci. 32, 267-287; Shaham, Y., Hope, B. T., 2005. The role of neuroadaptations in relapse to drug seeking Nat. Neurosci. 8, 1437-1439; Thomas, M. J., Kalivas, P. W., Shaham, Y., 2008. Neuroplasticity in the mesolimbic dopamine system and cocaine addiction. Br. J. Pharmacol. 154, 327-342).

Although there is a substantial body of evidence supporting a role for the excitatory amino acid neurotransmitter glutamate and its ligand-gated ionotropic receptors (i.e., N-methyl-D-aspartate (NMDA), a-amino-3-hydroxyl-5-methyl-4-isoxazole-propionate (AMPA), and kainic acid (KA) subtypes) in mediating addictive behaviors that dates back more than two decades (Gass, J. T., Olive, M. F., 2008. Glutamatergic substrates of drug addiction and alcoholism. Biochem. Pharmacol. 75, 218-265; Kalivas, P. W., 2004. Glutamate systems in cocaine addiction. Curr. Opin. Pharmacol. 4, 23-29; Tzschentke, T. M., Schmidt, W. J., 2003. Glutamatergic mechanisms in addiction. Mol. Psychiatry 8, 373-382; Wolf, M. E., 1998. The role of excitatory amino acids in behavioral sensitization to psychomotor stimulants. Prog. Neurobiol. 54, 679-720), it is only within the last decade or so that it has become apparent that metabotropic glutamate (mG1u) receptors are also involved in the neural mechanisms underlying drug addiction. Studies using pharmacological and genetic approaches have revealed clear evidence for a role of Group I (mGlu1 and mGluR5) receptors in regulating drug intake, reward, reinforcement, and reinstatement of drug-seeking behavior (Olive, M. F., 2009. Metabotropic glutamate receptor ligands as potential therapeutics for drug addiction. Curr. Drug Abuse Rev. 2, 83-98). However, Group I mGlu receptors also mediate cognitive processes such as learning and memory, behavioral flexibility, and extinction (Darrah, J. M., Stefani, M. R., Moghaddam, B., 2008. Interaction of N-methyl-D-aspartate and group 5 metabotropic glutamate receptors on behavioral flexibility using a novel operant set-shift paradigm. Behay. Pharmacol. 19, 225-234; Gass, J. T., Olive, M. F., 2009. Positive allosteric modulation of mGluR5 receptors facilitates extinction of a cocaine contextual memory. Biol. Psychiatry 65, 717-720; Moghaddam, B., 2004. Targeting metabotropic glutamate receptors for treatment of the cognitive symptoms of schizophrenia. Psychopharmacology (Berl) 174, 39-44; Shipe, W. D., Wolkenberg, S. E., Williams Jr., D. L., Lindsley, C. W., 2005. Recent advances in positive allosteric modulators of metabotropic glutamate receptors. Curr. Opin. Drug Discov. Dev. 8, 449-457; Simonyi, A., Schachtman, T. R., Christoffersen, G. R., 2005. The role of metabotropic glutamate receptor 5 in learning and memory processes. Drug News Perspect. 18, 353-361), and deficits in these forms of cognition are frequently observed in drug addicts.

Studies have shown that pharmacological antagonism of mGluR5 receptors reduces drug reward, reinforcement, and reinstatement (see, e.g., Cleva, R. M. and Olive, M. F. 2011. Positive allosteric modulators of Type 5 Metabotropic glutamate Receptors (mGluR5) and their therapeutic potential for the treatment of CNS disorders. Molecules 16, 2097-2106). For example, animal models of addiction have shown that mGluR5 antagonists, such as 2-methyl-6-(phenylethynyl)pyridine hydrochloride (MPEP) and 3-((2-methyl-4-thiazolyl)ethynyl)pyridine (MTEP), reduce self-administration of virtually all drugs of abuse (see, e.g., Olive, M. F. 2010).

Recent studies have focused on positive allosteric modulation (PAM) of the mGluR5 receptors for the treatment of CNS disorders, including schizophrenia, cognitive deficits associated with chronic drug use and deficits in extinction learning (Cleva, R. M. and Olive, M. F. 2011). Gass and Olive showed that the mGluR5 PAM 3-cyano-N-(1,3,-diphenyl-1H-pyrazol-5-yl)benzamide (CDPPB) enhances extinction of cocaine-induced conditioned place preference in rats (Gass, J. T. and Olive, M. F. 2009. Positive allosteric modulation of mGluR5 receptors facilitates extinction learning of a cocaine contextual memory. Biol. Psychiatry 65, 717-720). Cleva et al. showed that CDPPB reduces cocaine-seeking behavior following intravenous self-administration and deters the reacquisition of cocaine self-administration (Cleve et al. 2011. mGluR5 positive allosteric modulation enhances extinction learning following cocaine self-administration. Behavioral Neuroscience 125(1), 10-19.) Kufahl et al. showed that CDPPB improved extinction following methamphetamine self-administration and reduced reinstatement slightly (Kufahl et al. 2012. Positive allosteric modulation of mGluR5 accelerates extinction learning but not relearning following methamphetamine self-administration. Frontiers in Pharmacology 3, 1-14.

### SUMMARY OF THE INVENTION

The present invention provides pharmaceutical compositions comprising (a) a pharmaceutically effective amount of an mGluR5 positive allosteric modulator, derivative, prodrug or a pharmaceutically acceptable salt thereof; (b) a pharmaceutically effective amount of an agent selected from an NMDA partial agonist or a GlyT1 inhibitor, derivative, prodrug or a pharmaceutically acceptable salt thereof; and (c) a pharmaceutically acceptable carrier.

In some embodiments, the agent is an NMDA partial agonist. In other embodiments, the agent is a GlyT1 inhibitor.

In some embodiments, the positive allosteric modulator is LSN2463359 [N-(1-methylethyl)-5-(pyridin-4-ylethynyl)pyridine-2-carboxamide] or a derivative, prodrug or pharmaceutically acceptable salt thereof.

In some embodiments, the positive allosteric modulator is 3-cyano-N-(1,3-diphenyl-1H-pyrazol-5-yl)benzamide (CDPPB) or a derivative, prodrug or pharmaceutically acceptable salt thereof.

In some embodiments, the NMDA partial agonist is selected from the group consisting of (R)-4-Amino-3-isoxazolidone, 4-Amino-3-isoxazolidinone (D-cycloserine), the peptide sequence Thr-Pro-Pro-Thr-NH2 (GLYX 13), R-(+)-cis-4-methyl-3-amino-1-hydroxypyrolid-2-one (L-687,414), derivatives, prodrugs and pharmaceutically acceptable salt thereof.

In some embodiments, the GlyT1 inhibitor is selected from the group consisting of ALX 5407 ((R)-(N-3-(4′-fluorophenyl)-3-(4′-phenylphenoxy)propyl)sarcosine); SSR 504734 ((2-chloro-N-[(S)-phenyl[(2S)-piperidin-2-yl] methyl]-3-trifluoromethyl benzamide); SSR 103800, LY 2365109 N-[2-[4-(1,3-Benzodioxol-5-yl)-2-(1-,1-dimethylethyl)phenoxy]ethyl]-N-methylglycine; 2-methoxy-N-{[4-phenyl-1-(propylthio)piperidin-4-yl]methyl}benzamide; (2- amino-6-chloro-N-{(1S)-1-[4-phenyl-1-(propylthio)piperidin-4-yl] ethyl} benzamide; (1-phenyl-8-[(1S,2S)-2-phenylcyclohexyl]-1,3,8-triazaspiro[4.5]decan-4-one); 4, (8-[(1S,2R)-2-hydroxy-2-phenylcyclohexyl]-1-phenyl-1,3,8-triazaspiro[4.5]decan-4-one); ((4R)-4-phenyl-8-[(1S,2S)-2-phenylcyclohexyl]-2,8-diazaspiro[4.5]decan-1-one); ((4R)-8-[(1S,2R)-2-hydroxy-2-phenylcyclohexyl]-4-phenyl-2,8- diazaspiro[4.5]decan-1-one); (4-(4-fluorophenyl)-8-[1-(4-fluorophenyl)cyclohexyl]-2,8-diazaspiro[4.5]decan-1-one); 8, 4-(4-{[2-(cyclopropylmethoxy)-5-(methylsulfonyl)phenyl]carbonyl}piperazin-1-yl)-3-fluorobenzonitrile; (R)-4-[5-chloro-2-(4- methoxy-phenylsulfanyl)-phenyl]-2-methyl-piperazin-1-yl-acetic acid; (S)-1-{2-[3-(3-fluoro-phenylsulfanyl) biphenyl-4-yloxy]ethyl}pyrrolidine-2-carboxylic acid; ORG 25935 (cis-N-methyl-N-(6-methoxy-1-phenyl-1,2,3,4-tetrahydronaphthalen-2- ylmethyl)amino-methylcarboxylic acid); Org 24598 (N-Methyl-N-[(3R)-3-phenyl-3-[4-(tri-fluoromethyl)phenoxy]propyl]-glycine); Compound R-231857; derivatives, prodrugs and pharmaceutically acceptable salt thereof.

The present invention also provides methods for treating drug addiction and for preventing a drug relapse in a patient in need thereof. The methods comprise (a) administering to the patient a pharmaceutically effective amount of an mGluR5 positive allosteric modulator, derivative, prodrug or a pharmaceutically acceptable salt thereof; (b) administering to the patient a pharmaceutically effective amount of an NMDA partial agonist, derivative, prodrug or a pharmaceutically acceptable salt thereof.

The present invention also provides methods for treating drug addiction and preventing a drug relapse in a patient in need thereof. The methods comprise (a) administering to the patient a pharmaceutically effective amount of an mGluR5 positive allosteric modulator, derivative, prodrug or a pharmaceutically acceptable salt thereof; and (b) administering to the patient a pharmaceutically effective amount of glycine transporter type 1 inhibitor, derivative, prodrug or a pharmaceutically acceptable salt thereof.

In some embodiments, the methods further comprise (c) exposing the patient to a drug-related cue or context

In some embodiments, the drug addiction is to a drug selected from the group consisting of cocaine, heroin, methamphetamine, nicotine, opiates, amphetamines and alcohol. In some embodiments, the drug is selected from the group consisting of cocaine, heroin and alcohol. In some aspects of this embodiment, the drug is cocaine. In other aspects, the drug is heroin. In other aspects, the drug is alcohol.

In some embodiments, the positive allosteric modulator is LSN2463359 [N-(1-methylethyl)-5-(pyridin-4-ylethynyl)pyridine-2-carboxamide] or a derivative, prodrug or pharmaceutically acceptable salt thereof.

In some embodiments, the positive allosteric modulator is 3-cyano-N-(1,3-diphenyl-1H-pyrazol-5-yl)benzamide (CDPPB) or a derivative, prodrug or pharmaceutically acceptable salt thereof

In some embodiments, more than one positive allosteric modulator, derivative, prodrug or pharmaceutically acceptable salt thereof is administered to the patient.

In some embodiments, the NMDA partial agonist is selected from the group consisting of (R)-4-Amino-3-isoxazolidone, 4-Amino-3-isoxazolidinone (D-cycloserine), the peptide sequence Thr-Pro-Pro-Thr-NH2 (GLYX 13), R-(+)-cis-4-methyl-3-amino-1-hydroxypyrolid-2-one (L-687,414), derivatives, prodrugs and pharmaceutically acceptable salt thereof.

In some embodiments, the GlyT1 inhibitor is selected from the group consisting of ALX 5407 ((R)-(N-[3-(4′-fluorophenyl)-3-(4′-phenylphenoxy)propyl])sarcosine); SSR 504734 ((2-chloro-N-[(S)-phenyl[(2S)-piperidin-2-yl] methyl]-3-trifluoromethyl benzamide); SSR 103800, LY 2365109 N-[2-[4-(1,3-Benzodioxol-5-yl)-2-(1-,1-dimethylethyl)phenoxy]ethyl]-N-methylglycine; 2-methoxy-N-{[4-phenyl-1-(propylthio)piperidin-4-yl]methyl}benzamide; (2-amino-6-chloro-N-{(1S)-1-[4-phenyl-1-(propylthio)piperidin-4-yl] ethyl}benzamide; (1-phenyl-8- [(1S ,2S)-2-phenylcyclohexyl]-1,3,8-triazaspiro[4.5]decan-4-one); 4, (8- [(1S ,2R)-2-hydroxy-2-phenylcyclohexyl]-1-phenyl-1,3,8-triazaspiro[4.5]decan-4-one); ((4R)-4-phenyl-8-[(1S,2S)-2- phenylcyclohexyl]-2,8-diazaspiro[4.5]decan-1-one); ((4R)-8-[(1S,2R)-2-hydroxy-2-phenylcyclohexyl]-4-phenyl-2,8- diazaspiro[4.5]decan-1-one); (4-(4-fluorophenyl)-8-[1-(4-fluorophenyl)cyclohexyl]-2,8-diazaspiro[4.5]decan-1-one); 8, 4-(4-{[2-(cyclopropylmethoxy)-5-(methylsulfonyl)phenyl]carbonyl}piperazin-1-yl)-3-fluorobenzonitrile; (R)-4-[5-chloro-2-(4- methoxy-phenylsulfanyl)-phenyl]-2-methyl-piperazin-1-yl-acetic acid; (S)-1-{2-[3-(3-fluoro-phenylsulfanyl) biphenyl-4-yloxy]ethyl}pyrrolidine-2-carboxylic acid; ORG 25935 (cis-N-methyl-N-(6-methoxy-1-phenyl-1,2,3,4-tetrahydronaphthalen-2-ylmethyl)amino-methylcarboxylic acid); Org 24598 (N-Methyl-N-[(3R)-3-phenyl-3-[4-(tri-fluoromethyl)phenoxy]propyl]-glycine); Compound R-231857; derivatives, prodrugs and pharmaceutically acceptable salt thereof.

In some embodiments, the positive allosteric modulators are administered in a pharmaceutical composition. The pharmaceutically acceptable composition comprises the mGluR5 positive allosteric modulator, derivative, prodrug or a pharmaceutically acceptable salt thereof and a pharmaceutically acceptable carrier.

In some embodiments, the NMDA partial agonists are administered in a pharmaceutical composition. The pharmaceutically acceptable composition comprises the NMDA partial agonist, derivative, prodrug or a pharmaceutically acceptable salt thereof and a pharmaceutically acceptable carrier.

In some embodiments, the glycine transporter type 1 inhibitors are administered in a pharmaceutical composition. The pharmaceutically acceptable composition comprises the glycine transporter type 1 inhibitor, derivative, prodrug or a pharmaceutically acceptable salt thereof and a pharmaceutically acceptable carrier.

In some embodiments, the pharmaceutically acceptable composition further comprises an additional agent useful in treating drug addiction or preventing drug relapse in the patient.

In some embodiments, the drug-related cue or context is selected from the group consisting of a photograph of drug paraphernalia, actual drug paraphernalia, a video of individuals administering drugs or alcohol, a bottle of alcohol, smell of alcohol, mock drug-using environment and a mock bar.

In some embodiments of the present invention, the methods enhance the extinction reactivity to a drug-associated cue or context in the patient. In other embodiments, the methods reduce the motivation to resume drug use triggered by exposure to a drug-associated cue or context.

### DETAILED DESCRIPTION OF THE INVENTION

In order that the invention herein described may be fully understood, the following detailed description is set forth.

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

### Pharmaceutical Compositions

The present invention provides a pharmaceutical composition comprising a pharmaceutically effective amount of an mGluR5 positive allosteric modulator, derivative, prodrug or a pharmaceutically acceptable salt thereof; (b) a pharmaceutically effective amount of an agent selected from an NMDA partial agonist or a GlyT1 inhibitor, derivative, prodrug or a pharmaceutically acceptable salt thereof; and (c) a pharmaceutically acceptable carrier.

The term “pharmaceutically effective amount” refers to the amount of the mGluR5 positive allosteric modulator or agent required to confer a therapeutic effect on the patient treated.

The term “positive allosteric modulator” may also be referred to as “PAM.”

The term “mGluR5” refers to the Type-5 metabotropic glutamate receptor. A synonym for mGluR5 is “mGlu5”.

The term “mGluR5 positive allosteric modulator” refers to any exogenously administered compound or agent that directly or indirectly augments the activity of the mGluR5 receptor in the presence of the endogenous ligand (such as glutamate, L-serine O-phosphate (L-SOP), other endogenous ligands, other neurotransmitters, etc.) in a patient.

Suitable positive allosteric modulators of mGluR5 useful in the present invention include, but are not limited to, those disclosed in Azetidinyl oxadiazoles as potent mGluR5 positive allosteric modulators. Packiarajan M, Ferreira C G, Hong S P, White A D, Chandrasena G, Pu X, Brodbeck R M, Robichaud A J. Bioorg Med Chem Lett. 2012 Oct 15;22(20):6469-74; N-Aryl pyrrolidinonyl oxadiazoles as potent mGluR5 positive allosteric modulators; Packiarajan M, Mazza Ferreira C G, Hong S P, White A D, Chandrasena G, Pu X, Brodbeck R M, Robichaud A J. Bioorg Med Chem Lett. 2012 Sep 1;22(17):5658-62; Design and synthesis of substituted N-(1,3-diphenyl-1H-pyrazol-5-yl)benzamides as positive allosteric modulators of the metabotropic glutamate receptor subtype 5. Zou M F, Cao J, Rodriguez A L, Conn P J, Newman A H. Bioorg Med Chem Lett. 2011 May 1;21(9):2650-4; Discovery of novel positive allosteric modulators of the metabotropic glutamate receptor 5 (mG1u5); Varnes J G, Marcus A P, Mauger R C, Throner S R, Hoesch V, King M M, Wang X, Sygowski L A, Spear N, Gadient R, Brown D G, Campbell J B; Bioorg Med Chem Lett. 2011 Mar 1;21(5):1402-6; 4-aryl piperazine and piperidine amides as novel mGluR5 positive allosteric modulators; Xiong H, Brugel T A, Balestra M, Brown D G, Brush K A, Hightower C, Hinkley L, Hoesch V, Kang J, Koether G M, McCauley J P Jr, McLaren F M, Panko L M, Simpson T R, Smith R W, Woods J M, Brockel B, Chhajlani V, Gadient R A, Spear N, Sygowski L A, Zhang M, Arora J, Breysse N, Wilson J M, Isaac M, Slassi A, King M M; Bioorg Med Chem Lett. 2010 Dec 15; 20(24):7381-4; U.S. Pat. No. 8,163,775 (Addex Pharma SA), U.S. Pat. No. 8,030,331 (Addex Pharma SA), U.S. Pat. No. 7,834,035 (Addex Pharma SA), U.S. Pat. No. 8,034,806 (Vanderbilt University), U.S. Pat. No. 8,440,837 (H. Lundbeck A/S); and U.S. Patent Application Publications 20090082399 (Addex Pharma SA), 20090197897 (Addex Pharma SA), 20090215822 (Nikem Research SRL and Addex Pharma SA), 20100004284 (Addex Pharma SA), 20100081690 (Addex Pharma SA), 20120252838 (Eli Lilly And Company).

In some embodiments, the positive allosteric modulator is LSN2463359 [N-(1-methylethyl)-5-(pyridin-4-ylethynyl)pyridine-2-carboxamide] or a derivative, prodrug or pharmaceutically acceptable salt thereof. In some embodiments, the mGluR5 positive allosteric modulator is 3-cyano-N-(1,3-diphenyl-1H-pyrazol-5-yl)benzamide (CDPPB).

In some embodiments, more than one mGluR5 positive allosteric modulator, derivative, prodrug or pharmaceutically acceptable salt thereof is administered to the patient. In some aspects of this embodiment, two mGluR5 PAMs are administered to the patient. In embodiments in which two mGluR5 PAMS are administered, they can be LSN2463359 and CDPPB.

The term “NMDA partial agonist” refers to a partial agonist of the NMDA receptor. The NMDA receptor is a glutamate receptor and is the predominant molecular device for controlling synaptic plasticity and memory function.

Suitable NMDA partial agonists useful in the present invention include, but are not limited to, (R)-4-Amino-3-isoxazolidone, 4-Amino-3-isoxazolidinone (D-cycloserine), Peptide sequence Thr-Pro-Pro-Thr-NH2 (GLYX 13) and R-(+)-cis-4-methyl-3-amino-1-hydroxypyrolid-2-one (L-687,414). In some embodiments, the NMDA partial agonist is (R)-4-Amino-3-isoxazolidone, 4-Amino-3-isoxazolidinone (D-cycloserine). In other embodiments, the NMDA partial agonist is Peptide sequence Thr-Pro-Pro-Thr-NH2 (GLYX 13). In yet other embodiments, the NMDA partial agonist is R-(+)-cis-4-methyl-3-amino-1-hydroxypyrolid-2-one (L-687,414).

The terms “Glycine transporter type 1 inhibitor” and “GlyT1 inhibitor” refer to inhibitors of the glycine transporter type 1 receptor.

Suitable GlyT1 inhibitors useful in the present invention include, but are not limited to, those disclosed in U.S. Pat. No. 8,436,019 (Vanderbilt University), U.S. Pat. No. 8,431,700 (Vanderbilt University), U.S. Pat. No. 8,420,670 (Abbott Laboratories), U.S. Pat. No. 8,258,306 (Amgen Inc.), U.S. Pat. No. 8,211,933 (Vanderbilt University), U.S. Pat. No. 8,207,155 (Vanderbilt University), U.S. Pat. No. 8,183,244 (Amgen Inc.), U.S. Pat. No. 8,163,956 (Merck Sharp & Dohme Corp.), U.S. Pat. No. 7,947,714 (Merck Sharp & Dohme Corp.), U.S. Pat. No. 7,851,638 (Merck Sharp & Dohme Corp.), U.S. Pat. No. 7,825,135 (Merck Sharp & Dohme Corp.), U.S. Pat. No. 7,776,886 (Merck Sharp & Dohme Corp.), U.S. Pat. No. 7,745,642 (Glaxo Group Limited), U.S. Pat. No. 7,655,644 (Merck Sharp & Dohme Corp.), U.S. Pat. No. 7,626,056 (Merck Sharp & Dohme Corp.), U.S. Pat. No. 7,576,083 (Merck & Co., Inc.), U.S. Pat. No. 7,572,792 (Merck & Co., Inc.), U.S. Pat. No. 7,538,114 (Amgen Inc.), U.S. Pat. No. 6,784,299 (Pfizer Inc.), U.S. Pat. No. 6,710,071 (Pfizer Inc.), U.S. Pat. No. 6,566,550 (Pfizer Inc.), U.S. Pat. No. 6,506,780 (Pfizer Inc.). Other useful GlyT1 inhibitors include, but are not limited to, ALX 5407 ((R)-(N-[3-(4′-fluorophenyl)-3-(4′-phenylphenoxy)propyl])sarcosine); SSR 504734 ((2-chloro-N-[(S)-phenyl[(2S)-piperidin-2-yl]methyl]-3-trifluoromethyl benzamide); SSR 103800, LY 2365109 N-[2-[4-(1,3-Benzodioxol-5-yl)-2-(1-,1-dimethylethyl)phenoxy]ethyl]-N-methylglycine; 2-methoxy-N- {[4-phenyl-1-(propylthio)piperidin-4-yl]methyl}benzamide; (2-amino-6-chloro-N-{(1S)-1-[4-phenyl-1-(propylthio)piperidin-4-yl] ethyl} benzamide; (1-phenyl-8-[(1S,25)-2-phenylcyclohexyl]-1,3,8-triazaspiro[4.5]decan-4-one); 4, (8-[(1S ,2R)-2-hydroxy-2-phenylcyclohexyl]-1-phenyl-1,3,8-triazaspiro[4.5]decan-4-one); ((4R)-4-phenyl-8-[(1S,2S)-2- phenylcyclohexyl]-2,8-diazaspiro[4.5]decan-1-one); ((4R)-8-[(1S,2R)-2-hydroxy-2-phenylcyclohexyl]-4-phenyl-2,8-diazaspiro[4.5]decan-1-one); (4-(4-fluorophenyl)-8-[1-(4-fluorophenyl)cyclohexyl]-2,8-diazaspiro[4.5]decan-1-one); 8, 4-(4-{[2-(cyclopropylmethoxy)-5-(methylsulfonyl)phenyl]carbonyl}piperazin-1-yl)-3-fluorobenzonitrile; (R)-4-[5-chloro-2-(4- methoxy-phenylsulfanyl)-phenyl]-2-methyl-piperazin-1-yl-acetic acid; (S)-1-{2-[3-(3-fluoro-phenylsulfanyl) biphenyl-4-yloxy]ethyl}pyrrolidine-2-carboxylic acid; ORG 25935 (cis-N-methyl-N-(6-methoxy-1-phenyl-1,2,3,4-tetrahydronaphthalen-2- ylmethyl)amino-methylcarboxylic acid); Org 24598 (N-Methyl-N-[(3R)-3-phenyl-3-[4-(tri-fluoromethyl)phenoxy]propyl]-glycine); Compound R-231857; derivatives, prodrugs and pharmaceutically acceptable salt thereof

According to the present invention, “a pharmaceutically acceptable derivative or prodrug” includes, but is not limited to, pharmaceutically acceptable salts, esters, salts of such esters, or any other adduct or derivative that, upon administration to a patient in need thereof, is capable of providing, directly or indirectly, an mGluR5 positive allosteric modulator, an NMDA partial agonist or a GlyT1 inhibitor, or a biologically active metabolite or residue thereof As used herein, the term “biologically active metabolite or residue thereof” means that the metabolite or residue thereof is also an mGluR5 positive allosteric modulator, NMDA partial agonist or GlyT1 inhibitor.

A “pharmaceutically acceptable salt” means any non-toxic salt of an mGluR5 positive allosteric modulator, NMDA partial agonist or GlyT1 inhibitor that, upon administration to a patient, is capable of providing, either directly or indirectly, the mGluR5 positive allosteric modulator, NMDA partial agonist or GlyT1 inhibitor, or a biologically active metabolite or residue thereof.

Pharmaceutically acceptable salts are well known in the art. See, e.g., S. M. Berge, et al., J. Pharmaceutical Sciences, 1977, 66, 1-19, incorporated herein by reference. Pharmaceutically acceptable salts of the mGluR5 positive allosteric modulators, NMDA agonists and GlyT1 inhibitors include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art, such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the mGluR5 positive allosteric modulators. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

The term “pharmaceutically acceptable carrier” refers to a non-toxic carrier, adjuvant, or vehicle that does not destroy the pharmacological activity of the active ingredients in the composition of the invention. Pharmaceutically acceptable carriers, adjuvants or vehicles that may be used in the pharmaceutically acceptable compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutically acceptable compositions of the present invention may be administered orally, parenterally, by inhalation spray, topically, rectally, nasally, buccally, vaginally or via an implanted reservoir. The term “parenteral”, as used herein, includes subcutaneous, intravenous, intramuscular, intra-articular, intra-synovial, intrasternal, intrathecal, intrahepatic, intralesional and intracranial injection or infusion techniques. Preferably, the pharmaceutically acceptable compositions are administered orally, intraperitoneally or intravenously. Sterile injectable forms of the pharmaceutically acceptable compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically. Topically-transdermal patches may also be used.

For topical applications, the pharmaceutically acceptable compositions may be formulated in a suitable ointment containing the active component suspended or dissolved in one or more carriers. Carriers for topical administration of the mGluR5 positive allosteric modulators include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

Dosage levels of between about 0.01 and about 100 mg/kg body weight per day, preferably between 0.5 and about 75 mg/kg body weight per day and most preferably between about 1 and 50 mg/kg body weight per day of the active ingredient compounds are useful in a therapy for treating drug addiction or preventing a drug relapse in a patient in need thereof.

Typically, the pharmaceutically acceptable compositions of this invention will be administered from about 1 to 5 times per day or alternatively, as a continuous infusion. Or, alternatively, the compositions of the present invention may be administered in a pulsatile formulation. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active ingredient (w/w). Preferably, such preparations contain from about 20% to about 80% active ingredient.

In some embodiments, the pharmaceutically acceptable composition further comprises an additional agent useful in treating drug addiction or preventing drug relapse in the patient. The additional agent can be a drug for maintenance of abstinence, such as naltrexone, acamprosate, disulfiram for alcoholism; methadone, buprenorphine, suboxone for opiate addiction, nicotine replacement therapy, zyban, varenicline for nicotine additiction, and the like

When the compositions of this invention comprise a combination of an mGluR5 positive allosteric modulator, an agent selected from an NMDA partial agonist or a GlyT1 inhibitor, and one or more additional therapeutic agents, each ingredient should be present at dosage levels of between about 10% to 80% of the dosage normally administered in a monotherapy regime.

Upon improvement of a patient's condition, a maintenance dose of an mGluR5 positive allosteric modulator and an agent selected from an NMDA partial agonist or a GlyT1 inhibitor; composition; or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of addiction or relapse of symptoms.

As the skilled artisan will appreciate, lower or higher doses than those recited above may be required. Specific dosage and treatment regimens for any particular patient will depend upon a variety of factors, including the activity of the specific mGluR5 positive allosteric modulator, NMDA partial agonist or GlyT1 inhibitor employed, the age, body weight, general health status, sex, diet, time of administration, rate of excretion, drug combination, the severity and course of the disease, and the patient's disposition to the disease and the judgment of the treating physician.

Depending upon the particular addiction to be treated or relapse to be prevented, additional therapeutic agents, which are normally administered to treat or prevent that addiction, may also be present in the compositions of this invention.

The mGluR5 positive allosteric modulators, NMDA partial agonists and GlyT1 inhibitors of the present invention may also be co-administered with additional therapeutic agents to increase the effect of therapy or prophylaxis against relapse. When the mGluR5 positive allosteric modulators, NMDA partial agonists and GlyT1 inhibitors of the present invention are administered in combination therapies with additional therapeutic agents, they may be administered sequentially or concurrently to the patient. Alternatively, pharmaceutically acceptable compositions according to this invention comprise a combination of an mGluR5 positive allosteric modulator, an NMDA partial agonist or GlyT1 inhibitor and another therapeutic agent.

### Methods

The present invention provides a method for treating drug addiction in a patient in need thereof. The method comprises (a) administering to the patient a pharmaceutically effective amount of an mGluR5 positive allosteric modulator, derivative, prodrug or a pharmaceutically acceptable salt thereof; and (b) administering to the patient a pharmaceutically effective amount of an agent selected from an NMDA partial agonist or a GlyT1 inhibitor, derivative, prodrug or a pharmaceutically acceptable salt thereof

The present invention also provides a method for preventing a drug relapse in a patient in need thereof. This method comprises (a) administering to the patient a pharmaceutically effective amount of an mGluR5 positive allosteric modulator, derivative, prodrug or a pharmaceutically acceptable salt thereof; (b) administering to the patient a pharmaceutically effective amount of an agent selected from an NMDA partial agonist or a GlyT1 inhibitor, derivative, prodrug or a pharmaceutically acceptable salt thereof.

As used herein, the terms “treat”, “treatment” and “treating” refer to the reduction or amelioration of the progression and/or severity of drug addiction or relapse, or the amelioration of one or more symptoms (preferably, one or more discernible symptoms) of drug addiction or relapse resulting from the administration of one or more therapies (e.g., one or more therapeutic agents such as an mGluR5 positive allosteric modulator).

As used herein, the terms “prevent”, “prevention” and “preventing” refer to the reduction in the risk of a drug relapse.

In some embodiments, the drug addiction is to a drug selected from the group consisting of cocaine, heroin, methamphetamine, nicotine, opiates, amphetamines and alcohol. In some embodiments, the drug is selected from the group consisting of cocaine, heroin and alcohol. In some aspects of this embodiment, the drug is cocaine. In other aspects, the drug is heroin. In other aspects, the drug is alcohol.

The term “patient”, as used herein, means an animal, preferably a mammal, and most preferably a human.

In some embodiments, the agent is an NMDA partial agonist. In alternate embodiments, the agent is a GlyT1 inhibitor.

In some embodiments, the positive allosteric modulator and agent are administered in a pharmaceutical composition. The pharmaceutically acceptable composition comprises (a) the mGluR5 positive allosteric modulator, derivative, prodrug or a pharmaceutically acceptable salt thereof; (b) an agent selected from an NMDA partial agonist or a GlyT1 inhibitor, derivative, prodrug or a pharmaceutically acceptable salt thereof; and (c) a pharmaceutically acceptable carrier. In some embodiments, the positive allosteric modulator and agent are administered in separate pharmaceutical compositions.

In some embodiments, the method further comprises (c) exposing the patient to a drug-related cue or context. Any drug-related cue or context may be used, including, but not limited to, a photograph of drug paraphernalia, actual drug paraphernalia, a video of individuals administering drugs or alcohol, a bottle of alcohol, smell of alcohol, mock drug-using environment, smell of cigarette smoke, or a mock bar.

In some embodiments of the present invention, the methods enhance the extinction reactivity to a drug-associated cue or context in the patient. As used herein, the term “extinction” refers to the decline in magnitude, frequency or both of a conditioned response either within a single-extinction training session or over successive extinction training sessions conducted on a routine basis (e.g., daily or weekly).

In other embodiments, the methods reduce the motivation to resume drug use triggered by exposure to a drug-associated cue or context.

In some embodiments, the present invention provides a method for enhancing extinction reactivity to a drug-associated cue or context in the patient. This method comprises (a) administering to the patient a pharmaceutically effective amount of an mGluR5 positive allosteric modulator, derivative, prodrug or a pharmaceutically acceptable salt thereof, or a pharmaceutical composition thereof and a pharmaceutically acceptable carrier; (b) administering to the patient a pharmaceutically effective amount of an agent selected from an NMDA partial agonist or a GlyT1 inhibitor, derivative, prodrug or a pharmaceutically acceptable salt thereof, or a pharmaceutical composition thereof and a pharmaceutically acceptable carrier.

In some embodiments, this method further comprises (c) exposing the patient to a drug-related cue or context. The mGluR5 PAM, NMDA partial agonist, GlyT1 inhibitor or composition thereof may be any described herein.

In some embodiments, the present invention provides a method for reducing the motivation to resume drug use triggered by exposure to a drug-associated cue or context. This method comprises (a) administering to the patient a pharmaceutically effective amount of an mGluR5 positive allosteric modulator, derivative, prodrug or a pharmaceutically acceptable salt thereof, or a pharmaceutical composition thereof and a pharmaceutically acceptable carrier; and (b) administering to the patient a pharmaceutically effective amount of an agent selected from an NMDA partial agonist or a GlyT1 inhibitor, derivative, prodrug or a pharmaceutically acceptable salt thereof, or a pharmaceutical composition thereof and a pharmaceutically acceptable carrier.

In some embodiments, this method further comprises (c) exposing the patient to a drug-related cue or context. The mGluR5 PAM, NMDA partial agonist, GlyT1 inhibitor or composition thereof may be any described herein.

It should be understood that the method steps may be performed in any order. For example, the mGluR5 positive allosteric modulator, derivative, prodrug or a pharmaceutically acceptable salt thereof, or pharmaceutically acceptable composition thereof, may be administered prior to administering the NMDA partial agonist or GlyT1 inhibitor, derivative, prodrug or a pharmaceutically acceptable salt thereof, or pharmaceutically acceptable composition thereof. Alternatively, the mGluR5 positive allosteric modulator, derivative, prodrug or a pharmaceutically acceptable salt thereof, or pharmaceutically acceptable composition thereof may be administered subsequent to administering the NMDA partial agonist or GlyT1 inhibitor, derivative, prodrug or a pharmaceutically acceptable salt thereof, or pharmaceutically acceptable composition thereof

In some embodiments, the mGluR5 positive allosteric modulator, NMDA partial agonist or GlyT1 inhibitor may be administered prior to exposing the patient to a drug-related cue or context. In some embodiments, the mGluR5 positive allosteric modulator, NMDA partial agonist or GlyT1 inhibitor are administered both prior and subsequent to exposing the patient to a drug-related cue or context.

The present invention also provides the use of an mGluR5 positive allosteric modulator, derivative, prodrug or pharmaceutically acceptable salt thereof, or a pharmaceutical composition thereof, and an agent selected from an NMDA partial agonist or a GlyT1 inhibitor, derivative, prodrug or a pharmaceutically acceptable salt thereof, or a pharmaceutical composition thereof, in the manufacture of a medicament for treating drug addiction or preventing a drug relapse. In some embodiments, the mGluR5 positive allosteric modulator and the agent, derivative, prodrug or a pharmaceutically acceptable salt thereof, or pharmaceutically acceptable composition thereof, are to be administered during cue exposure therapy. The mGluR5 positive allosteric modulator or agent, derivative, prodrug or a pharmaceutically acceptable salt thereof or pharmaceutically acceptable composition thereof, may be any described above.

As used herein, the term “cue exposure therapy” refers to a therapy based on the premise that repeated exposure of a patient to cue associated with drug use in a safe and controlled environment will desensitize the patient to these cues and therefore reduce the pathological thought and behavioral patterns associated with drug use.

While particular materials, formulations, operational sequences, process parameters, and end products have been set forth to describe and exemplify this invention, they are not intended to be limiting. Rather, it should be noted by those ordinarily skilled in the art that the written disclosures are exemplary only and that various other alternatives, adaptations, and modifications may be made within the scope of the present invention. Accordingly, the present invention is not limited to the specific embodiments illustrated herein, but is limited only by the following claims.

What is claimed is:

1. A pharmaceutical composition comprising
(a) a pharmaceutically effective amount of an mGluR5 positive allosteric modulator, derivative, prodrug or a pharmaceutically acceptable salt thereof;
(b) a pharmaceutically effective amount of an agent selected from an NMDA partial agonist or a GlyT1 inhibitor, derivative, prodrug or a pharmaceutically acceptable salt thereof and
(c) a pharmaceutically acceptable carrier.

2. The pharmaceutical composition according to claim 1, wherein the agent is an NMDA partial agonist.

3. The pharmaceutical composition according to claim 1, wherein the agent is a GlyT1 inhibitor.

4. The pharmaceutical composition according to claim 1, wherein the mGluR5 positive allosteric modulator is LSN2463359 [N-(1-methylethyl)-5-(pyridin-4-ylethynyl)pyridine-2-carboxamide] or a derivative, prodrug or pharmaceutically acceptable salt thereof.

5. The pharmaceutical composition according to claim 1, wherein the mGluR5 positive allosteric modulator is 3-cyano-N-(1,3-diphenyl-1H-pyrazol-5-yl)benzamide (CDPPB) or a derivative, prodrug or pharmaceutically acceptable salt thereof.

6. A method for treating drug addiction in a patient in need thereof comprising:
(a) administering to the patient a pharmaceutically effective amount of an mGluR5 positive allosteric modulator, derivative, prodrug or a pharmaceutically acceptable salt thereof, or a pharmaceutical composition comprising the mGluR5 positive allosteric modulator, derivative, prodrug or a pharmaceutically acceptable salt thereof and a pharmaceutically acceptable carrier; and
(b) administering to the patient a pharmaceutically effective amount of an agent selected from an NMDA partial agonist or a GlyT1 inhibitor, derivative, prodrug or a pharmaceutically acceptable salt thereof, or a pharmaceutical composition comprising the agent, derivative, prodrug or a pharmaceutically acceptable salt thereof and a pharmaceutically acceptable carrier.

7. A method for preventing a drug relapse in a patient in need thereof comprising:
(a) administering to the patient a pharmaceutically effective amount of an mGluR5 positive allosteric modulator, derivative, prodrug or a pharmaceutically acceptable salt thereof, or a pharmaceutical composition comprising the mGluR5 positive allosteric modulator, derivative, prodrug or a pharmaceutically acceptable salt thereof and a pharmaceutically acceptable carrier; and
(b) administering to the patient a pharmaceutically effective amount of an agent selected from an NMDA partial agonist or a GlyT1 inhibitor, derivative, prodrug or a pharmaceutically acceptable salt thereof, or a pharmaceutical composition comprising the agent, derivative, prodrug or a pharmaceutically acceptable salt thereof and a pharmaceutically acceptable carrier.

8. The method according to claim 6 or 7, wherein the agent is an NMDA partial agonist.

9. The method according to claim 6 or 7, wherein the agent is a GlyT1 inhibitor.

10. The method according to claim 6 or 7, further comprising (c) exposing the patient to a drug-related cue or context.

11. The method according to claim 6 or 7, wherein steps (a) and (b) enhance the extinction reactivity to a drug-associated cue or context in the patient.

12. The method according to claim 6 or 7, wherein steps (a) and (b) reduce the motivation to resume drug use triggered by exposure to a drug-associated cue or context.

13. The method according to claim 6 or 7, wherein the drug addiction is to cocaine, heroin, methamphetamine or alcohol

14. The method according to claim 6 or 7, wherein the mGluR5 positive allosteric modulator is LSN2463359 [N-(1-methylethyl)-5-(pyridin-4-ylethynyl)pyridine-2-carboxamide] or a derivative, prodrug or pharmaceutically acceptable salt thereof.

15. The method according to claim 6 or 7, wherein the mGluR5 positive allosteric modulator is 3-cyano-N-(1,3-diphenyl-1H-pyrazol-5-yl)benzamide (CDPPB) or a derivative, prodrug or pharmaceutically acceptable salt thereof.

16. The method according to claim 6 or 7, wherein the pharmaceutically acceptable composition further comprises an additional agent useful in treating drug addiction or preventing drug relapse in the patient.

17. The method according to claim 6 or 7, wherein the drug-related cue or context is selected from the group consisting of a photograph of drug paraphernalia, actual drug paraphernalia, a video of individuals administering drugs or alcohol, a bottle of alcohol, smell of alcohol, a mock drug-using environment and a mock bar.

18. The use of an G1uR5 positive allosteric modulator and an agent, derivative, prodrug or a pharmaceutically acceptable salt thereof in the manufacture of a medicament for treating drug addiction, wherein the agent is selected from an NMDA partial agonist or a GlyT1 inhibitor.

19. The use of an GluR5 positive allosteric modulator and an agent, derivative, prodrug or a pharmaceutically acceptable salt thereof in the manufacture of a medicament for preventing a drug relapse, wherein the agent is selected from an NMDA partial agonist or a GlyT1 inhibitor.

20. The use according to claim 18 or 19, wherein the agent is an NMDA partial agonist.

21. The use according to claim 18 or 19, wherein the agent is a GlyT1 inhibitor.

22. The use according to claim 18 or 19, wherein GluR5 positive allosteric modulator and agent, derivative, prodrug or a pharmaceutically acceptable salt thereof are to be administered during cue exposure therapy.
